(19) United States
(12) Patent Application Publication
(10) Pub. No.: US 2024/0074625 A1
(43) Pub. Date: Mar. 7, 2024

(54) HYGIENE PRODUCT DISPENSER WITH A PLURALITY OF PRODUCTS

(71) Applicant: OFER SHECHTER LTD., Hadar-Am (IL)

(72) Inventor: Ofer SHECHTER, Hadar-Am (IL)

(21) Appl. No.: 18/027,118

(22) PCT Filed: Sep. 20, 2021
(86) PCT No.: PCT/IL2021/051141

§ 371 Date: Mar. 19, 2023

(30) Foreign Application Priority Data
Sep. 22, 2020 (IL) 277525

(51) Int. Cl. A47K 10/22; A47K 10/42

(52) U.S. Cl. CPC A47K 10/22; A47K 10/421; A47K 2010/3266

(57) ABSTRACT

A dispenser for containing a plurality of products, the dispenser comprising at least one adapter and at least one inner compartment. The adapter is configured to securely hold at least one first product, with the at least one first product being holdable directly to the adapter. The at least one inner compartment is configured to contain at least one second product, the inner compartment at least partially within a lumen of said adapter. The at least one adapter and the inner compartment being unitary or separable.

### FIELD OF THE INVENTION

The present invention generally pertains to a system and method for delivering a plurality of products, for example, hygiene products, from a single dispenser.

### BACKGROUND OF THE INVENTION

It is often useful to have at hand more than one type of cleansing material, for example, wet disposable wiping product such as baby wipes and dry disposable wiping product such as toilet paper or kitchen towels, the dry disposable wiping product to mop up the larger portions of a dirtied object or to mop up spilled fluid and the wet disposable wiping product to cleanse the remaining dirt or fluid from the object.

It can be inconvenient to have to find where the various cleansing materials are stored and then to find places to put both types of cleansing material.

It is therefore a long felt need to provide a system which does not require a plurality of dispensers for the plurality of products.

### SUMMARY OF THE INVENTION

It is another object of the present invention to disclose a dispenser for containing a plurality of products characterized by:
at least one adapter, said at least one adapter configured to securely hold at least one first product; said at least one first product being holdable directly to said adapter; and
at least one inner compartment configured to contain at least one second product, said at least one inner compartment at least partially within a lumen of said adapter
said at least one adapter and said at least one inner compartment being unitary or separable.

It is another object of the present invention to disclose the dispenser as described above, wherein said at least one first product is selected from a group consisting of dry disposable wiping product, wet disposable wiping product, and any combination thereof.

It is another object of the present invention to disclose the dispenser as described above, wherein said at least one second product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
a. said cleansing agent is selected from a group consisting of a liquid, a semi-liquid and a gel; and
b. said cleansing agent is selected from a group consisting of alcohol, an alcohol gel, an antiseptic, hand cleanser and any combination thereof.

It is another object of the present invention to disclose the dispenser as described above, wherein said at least one inner compartment comprises a reversibly closable lid, said closable lid being sealably closable; at least one of the following being true:
a. a closure of said closable lid is selected from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and in said at least one inner compartment overlap, and any combination thereof;
b. said closable lid is flexible, semi-rigid, rigid, and any combination thereof;
c. a location of an external side of said closable lid is selected from a group consisting of proud of said at least one inner compartment, flush with said at least one inner compartment and recessed below an external side of said at least one inner compartment;
d. said closable lid comprises a material selected from a group consisting of metal, plastic, silicone, cardboard, glass, paper, cork and any combination thereof;
e. said closable lid is selected from a group consisting of tamper-evident, child-proof and any combination thereof;
f. said closable lid is selected from a group consisting of a dispensing lid, a spray lid and any combination thereof; and
g. said dispensing lid comprises a member of a group consisting of a pour spout, a trigger, a sprayer cap, a pump, a measuring attachment, a sifting device, a valve, an actuator and any combination thereof.

It is another object of the present invention to disclose the dispenser as described above, additionally comprising a hollow lumen configured by means of shape and size to enable said dispenser to be emplaced on either a hanging implement or a support; at least one of the following being true:
a. said hanging implement is selected from a group consisting of be a horizontal rod, a wire, a rope, a string, a hook, a shackle, a suction cup holder and any combination thereof; and
b. said support is selected from a group consisting of a stand, a leg and any combination thereof.

It is another object of the present invention to disclose the dispenser as described above, wherein a material of said dispenser is selected from a group consisting of metal, plastic, silicone, cardboard, glass, paper and any combination thereof.

It is another object of the present invention to disclose the dispenser as described above, wherein at least one of the following is true:
a. said metal comprises aluminum, iron and any combination thereof;
b. a material of said plastic is selected from a group consisting of rigid, semi-rigid, flexible and any combination thereof; and
c. said plastic is selected from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

It is another object of the present invention to disclose the dispenser as described above, wherein at least one of the following is true:
a. a member of a group consisting of said at least one adapter, said at least one compartment and any combination thereof is refillable; and
b. a member of a group consisting of said at least one adapter, said at least one compartment and any combination thereof is separable from said dispenser.

It is another object of the present invention to disclose the dispenser as described above, additionally comprising a pipe with a lumen, said pipe providing fluid connection between said at least one inner compartment and an exterior of said device.

It is another object of the present invention to disclose the dispenser as described above, wherein at least one of the following is true:
a. said pipe is selected from a group consisting of flexible, semi-flexible, rigid and any combination thereof;
b. an external opening of said pipe is selected from a group consisting of flush with the at least one inner compartment, extend beyond the top of the at least one inner compartment, and be recessed into the at least one inner compartment;
c. said pipe is either fixed relative to the at least one inner compartment or movable relative to the at least one inner compartment;
d. said pipe, when said lid is closed, is flush with the at least one inner compartment and, when said lid is open, extends beyond the top of the at least one inner compartment;
e. said pipe is extendable beyond the top edge of the at least one inner compartment; and
f. the pipe comprises a member of a group consisting of a lip pullable by a user, an accordion foldable section at its exit end, be attached to the lid such that opening and closing the lid reversibly pulls the end of the pipe, a flexible portion such that pushing on it extrudes the pipe, and any combination thereof.

It is another object of the present invention to disclose the dispenser as described above, wherein an exit end of said pipe is closeable by a conventional sealing closure selected from a group consisting of a cork, a clip-on fitting, a screw-on fitting, a friction-fit fitting, a slide-over fitting, a pinch fitting and any combination thereof.

It is another object of the present invention to disclose the dispenser as described above, wherein at least one of the following is true:
a. said sealing closure is selected from a group consisting of tamper-evident, child-proof and any combination thereof;
b. said sealing closure is selected from a member of a group consisting of a pour spout, a trigger, a sprayer cap, a pump, a thumb stop a measuring attachment, a valve, an actuator and any combination thereof;
c. at least one of said pipe and said sealing closure is configured such that at least one of closure of the exit and pressure on the pipe forces contents of the pipe proximally; and
d. said sealing closure is selected from a group consisting of tamper-evident, child-proof and any combination thereof.

It is another object of the present invention to disclose the dispenser as described above, additionally comprising at least one wing configured by means of size and shape to prevent sliding between said adapter and a member of a group consisting of said first product, said inner compartment and any combination thereof; said at least one wing having a configuration selected from a group consisting of longitudinal corrugations, longitudinal ridges, transverse corrugations, transverse ridges, compressible pillows and any combination thereof.

It is another object of the present invention to disclose a method for dispensing a plurality of products, comprising steps of:
providing a dispenser for containing said plurality of products characterized by:
   at least one adapter, said at least one adapter configured to securely hold at least one first product; said at least one first product being holdable directly to said adapter; and
   at least one inner compartment configured to contain at least one second product, said at least one inner compartment at least partially within a lumen of said adapter;
   said at least one adapter and said at least one inner compartment being unitary or separable;
emplacing said at least one first product on said adapter;
emplacing said at least one second product in said at least one inner compartment.

It is another object of the present invention to disclose the method as described above, additionally comprising a step of selecting said at least one first product from a group consisting of dry disposable wiping product, wet disposable wiping product, and any combination thereof.

It is another object of the present invention to disclose the method as described above, additionally comprising a step of selecting said at least one second product from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
a. selecting said cleansing agent from a group consisting of a liquid, a semi-liquid and a gel; and
b. selecting said cleansing agent from a group consisting of alcohol, an alcohol gel, an antiseptic, hand cleanser and any combination thereof.

It is another object of the present invention to disclose the method as described above, additionally comprising a step of providing said at least one inner compartment comprising a reversibly closable lid, said closable lid being sealably closable; at least one of the following being true:
a. selecting a closure of said closable lid from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and in said at least one inner compartment overlap, and any combination thereof;
b. selecting a material of said closable lid from a group consisting of flexible, semi-rigid, rigid, and any combination thereof;
c. selecting a location of an external side of said closable lid from a group consisting of proud of said at least one inner compartment, flush with said at least one inner compartment and recessed below an external side of said at least one inner compartment;
d. providing said closable lid comprising a material selected from a group consisting of metal, plastic, silicone, cardboard, glass, paper, cork and any combination thereof;
e. said closable lid is selected from a group consisting of tamper-evident, child-proof and any combination thereof;
f. selecting said closable lid from a group consisting of a dispensing lid, a spray lid and any combination thereof; and
g. providing said dispensing lid comprising a member of a group consisting of a pour spout, a trigger, a sprayer cap, a pump, a measuring attachment, a sifting device, a valve, an actuator and any combination thereof.

It is another object of the present invention to disclose the method as described above, additionally comprising a step of providing said dispenser with a hollow lumen configured by means of shape and size to enable said dispenser to be emplaced on either a hanging implement or a support; at least one of the following being true:
a. selecting said hanging implement from a group consisting of be a horizontal rod, a wire, a rope, a string, a hook, a shackle, a suction cup holder and any combination thereof; and
b. selecting said support from a group consisting of a stand, a leg and any combination thereof.

It is another object of the present invention to disclose the method as described above, additionally comprising a step of selecting a material of said dispenser from a group consisting of metal, plastic, silicone, cardboard, glass, paper and any combination thereof. It is another object of the present invention to disclose the method as described above, additionally comprising at least one of the following steps:
a. comprising said metal of aluminum, iron and any combination thereof;
b. selecting a material of said plastic from a group consisting of rigid, semi-rigid, flexible and any combination thereof; and
c. selecting said plastic from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

It is another object of the present invention to disclose the method as described above, additionally comprising at least one of the following steps:
a. providing a member of a group consisting of said at least one adapter, said at least one compartment and any combination thereof refillable; and
b. providing a member of a group consisting of said at least one adapter, said at least one compartment and any combination thereof separable from said dispenser.

It is another object of the present invention to disclose the method as described above, additionally comprising providing said at least one inner compartment comprising a pipe with a lumen, said pipe providing fluid connection between said at least one inner compartment and an exterior of said device.

It is another object of the present invention to disclose the method as described above, additionally comprising at least one of the following steps:
a. selecting said pipe from a group consisting of flexible, semi-flexible, rigid and any combination thereof;
b. selecting an external opening of said pipe from a group consisting of flush with the at least one inner compartment, extend beyond the top of the at least one inner compartment, and be recessed into the at least one inner compartment;
c. providing said pipe either fixed relative to the at least one inner compartment or movable relative to the at least one inner compartment;
d. providing said pipe, when said lid is closed, flush with the at least one inner compartment and, when said lid is open, extends beyond the top of the at least one inner compartment;
e. providing said pipe extendable beyond the top edge of the at least one inner compartment; and
f. providing the pipe comprising a member of a group consisting of a lip pullable by a user, an accordion foldable section at its exit end, be attached to the lid such that opening and closing the lid reversibly pulls the end of the pipe, a flexible portion such that pushing on it extrudes the pipe, and any combination thereof.

It is another object of the present invention to disclose the method as described above, additionally comprising a step of closing an exit end of said pipe by a conventional sealing closure selected from a group consisting of a cork, a clip-on fitting, a screw-on fitting, a friction-fit fitting, a slide-over fitting, a pinch fitting and any combination thereof.

It is another object of the present invention to disclose the method as described above, additionally comprising at least one of the following steps:
a. selecting said sealing closure from a group consisting of tamper-evident, child-proof and any combination thereof;
b. selecting said sealing closure from a member of a group consisting of a pour spout, a trigger, a sprayer cap, a pump, a thumb stop a measuring attachment, a valve, an actuator and any combination thereof;
c. configuring at least one of said pipe and said sealing closure such that at least one of closure of the exit and pressure on the pipe forces contents of the pipe proximally; and
d. selecting said sealing closure from a group consisting of tamper-evident, child-proof and any combination thereof.

It is another object of the present invention to disclose the method as described above, additionally comprising a step of providing said dispenser comprising at least one wing configured by means of size and shape to prevent sliding between said adapter and a member of a group consisting of said first product, said inner compartment and any combination thereof; said at least one wing having a configuration selected from a group consisting of longitudinal corrugations, longitudinal ridges, transverse corrugations, transverse ridges, compressible pillows and any combination thereof.

It is another object of the present invention to disclose a dispenser for containing a plurality of products comprising:
at least one adapter, said at least one adapter configured to securely hold at least one first product; said at least one first product being holdable in a manner selected from a group consisting of directly to said adapter, in an external compartment and any combination thereof; and
at least one inner compartment configured to contain at least one second product, said at least one inner compartment at least partially within a lumen of said adapter.

It is another object of the present invention to disclose the dispenser as described above, wherein said at least one first product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
c. said cleansing agent is selected from a group consisting of a liquid, a semi-liquid and a gel; and
d. said cleansing agent is selected from a group consisting of alcohol, an alcohol gel, an antiseptic, hand cleanser and any combination thereof.

It is another object of the present invention to disclose the dispenser as described above, wherein said at least one second product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
a. said cleansing agent is selected from a group consisting of a liquid, a semi-liquid and a gel; and
b. said cleansing agent is selected from a group consisting of alcohol, an alcohol gel, an antiseptic, hand cleanser and any combination thereof.

It is another object of the present invention to disclose the dispenser as described above, wherein said at least one adapter and said at least one inner compartment are arranged in a manner selected from a group consisting of concentrically and linearly.

It is another object of the present invention to disclose the dispenser as described above, wherein a member of a group consisting of said external compartment, said at least one inner compartment and any combination thereof comprises a reversibly closable lid, said closable lid being sealably closable; at least one of the following being true:
a. a closure of said closable lid is selected from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and in said at least one inner compartment overlap, and any combination thereof;
b. said closable lid is flexible, semi-rigid, rigid, and any combination thereof;
c. a location of an external side of said closable lid is selected from a group consisting of proud of said at least one inner compartment, flush with said at least one inner compartment and recessed below an external side of said at least one inner compartment;
d. said closable lid comprises a material selected from a group consisting of metal, plastic, silicone, cardboard, glass, paper, cork and any combination thereof;
e. said closable lid is selected from a group consisting of tamper-evident, child-proof and any combination thereof;
f. said closable lid is selected from a group consisting of a dispensing lid, a spray lid and any combination thereof; and
g. said dispensing lid comprises a member of a group consisting of a pour spout, a trigger, a sprayer cap, a pump, a measuring attachment, a sifting device, a valve, an actuator and any combination thereof.

It is another object of the present invention to disclose the dispenser as described above, additionally comprising a hollow lumen configured by means of shape and size to enable said dispenser to be emplaced on either a hanging implement or a support; at least one of the following being true:
a. said hanging implement is selected from a group consisting of be a horizontal rod, a wire, a rope, a string, a hook, a shackle, a suction cup holder and any combination thereof; and
b. said support is selected from a group consisting of a stand, a leg and any combination thereof.

It is another object of the present invention to disclose the dispenser as described above, wherein a material of said dispenser is selected from a group consisting of metal, plastic, silicone, cardboard, glass, paper and any combination thereof; at least one of the following being true:
a. said metal comprises aluminum, iron and any combination thereof;
b. a material of said plastic is selected from a group consisting of rigid, semi-rigid, flexible and any combination thereof; and
c. said plastic is selected from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

It is another object of the present invention to disclose the dispenser as described above, wherein at least one of the following is true:
a. a member of a group consisting of said at least one adapter, said external compartment, said at least one inner compartment and any combination thereof is refillable; and
b. a member of a group consisting of said at least one adapter, said external compartment, said at least one inner compartment and any combination thereof is separable from said dispenser.

It is another object of the present invention to disclose the dispenser as described above, wherein a piped compartment is selected from a group consisting of said external compartment, said at least one inner compartment and any combination thereof; said piped compartment comprising a pipe with a lumen, said pipe providing fluid connection between said piped compartment and an exterior of said device; at least one of the following being true:
a. said pipe is selected from a group consisting of flexible, semi-flexible, rigid and any combination thereof;
b. an external opening of said pipe is selected from a group consisting of flush with the piped compartment, extending beyond a top of the piped compartment, and recessed into the piped compartment;
c. said pipe is either fixed relative to the piped compartment or movable relative to the piped compartment;
d. an exit end of said pipe, when said lid is closed, is flush with the piped compartment and, when said lid is open, said pipe extends beyond the top of the piped compartment;
e. said pipe is extendable beyond the top edge of the piped compartment; and
f. the pipe comprises a member of a group consisting of a lip pullable by a user, an accordion foldable section at its exit end, attachment to the lid such that opening and closing the lid reversibly pulls the exit end of the pipe, a flexible portion such that pulling or pushing on it extrudes the pipe, and any combination thereof.

It is another object of the present invention to disclose the dispenser as described above, wherein the exit end of said pipe is closeable by a conventional sealing closure selected from a group consisting of a cork, a clip-on fitting, a screw-on fitting, a friction-fit fitting, a slide-over fitting, a pinch fitting and any combination thereof; at least one of the following being true:
a. said sealing closure is selected from a group consisting of tamper-evident, child-proof and any combination thereof;
b. said sealing closure is selected from a member of a group consisting of a pour spout, a trigger, a sprayer cap, a pump, a thumb stop a measuring attachment, a valve, an actuator and any combination thereof;
c. at least one of said pipe and said sealing closure is configured such that at least one of closure of the exit and pressure on the pipe forces contents of the pipe proximally; and
d. said sealing closure is selected from a group consisting of tamper-evident, child-proof and any combination thereof.

It is another object of the present invention to disclose the dispenser as described above, additionally comprising at least one wing configured by means of size and shape to prevent sliding between said adapter and a member of a group consisting of said roll of dry disposable wiping product, said external compartment, said inner compartment and any combination thereof; said at least one wing having a configuration selected from a group consisting of longitudinal corrugations, longitudinal ridges, transverse corrugations, transverse ridges, compressible pillows and any combination thereof.

It is another object of the present invention to disclose a method of providing a dispenser for a plurality of products comprising steps of:
providing a dispenser for containing said plurality of products comprising:
   at least one adapter, said at least one adapter configured to securely hold at least one first product, said at least one first product being holdable in a manner selected from a group consisting of directly to said adapter, in an external compartment and any combination thereof; and
   at least one inner compartment configured to contain at least one second product, said at least one inner compartment at least partially within a lumen of said adapter;
for said at least one first product being securely holdable directly to said adapter, emplacing said at least one first product to said at least one adapter;
for said at least one first product being holdable in an external compartment, emplacing said external compartment to said at least one adapter and emplacing in said external compartment said at least one first product;
emplacing said at least one second product in said at least one inner compartment.

It is another object of the present invention to disclose the method as described above, additionally comprising a step of selecting said at least one first product from of a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; additionally comprising at least one of the following steps:
e. selecting said cleansing agent from a group consisting of a liquid, a semi-liquid and a gel; and
f. selecting said cleansing agent from a group consisting of alcohol, an alcohol gel, an antiseptic, hand cleanser and any combination thereof.

It is another object of the present invention to disclose the method as described above, additionally comprising a step of selecting said at least one second product from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; additionally comprising at least one of the following steps:
a. selecting said cleansing agent from a group consisting of a liquid, a semi-liquid and a gel; and
b. selecting said cleansing agent from a group consisting of alcohol, an alcohol gel, an antiseptic, hand cleanser and any combination thereof.

It is another object of the present invention to disclose the method as described above, additionally comprising a step of selecting a manner of arranging said at least one adapter and said at least one inner compartment from a group consisting of concentrically and linearly.

It is another object of the present invention to disclose the method as described above, additionally comprising a step of providing a member of a group consisting of said external compartment, said at least one inner compartment and any combination thereof comprising a reversibly closable lid, said closable lid being sealably closable; additionally comprising at least one of the following steps:
a. selecting a closure of said closable lid from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and said at least one inner compartment overlap, and any combination thereof;
b. providing said closable lid to be flexible, semi-rigid, rigid, and any combination thereof;
c. selecting a location of an external side of said lid from a group consisting of proud of said at least one inner compartment, flush with said at least one inner compartment and recessed below an external side of said at least one inner compartment;
d. providing said closable lid comprising a material selected from a group consisting of metal, plastic, silicone, cardboard, glass, paper, cork and any combination thereof;
e. selecting said closable lid to be tamper-evident, child-proof and any combination thereof;
f. selecting said closable lid from a member of a group consisting of a dispensing lid, a spray lid and any combination thereof; and
g. providing said dispensing lid comprising a member of a group consisting of a pour spout, a trigger, a sprayer cap, a pump, a measuring attachment, a sifting device, a valve, an actuator and any combination thereof.

It is another object of the present invention to disclose the method as described above, additionally comprising a step of providing said dispenser with a lumen configured by means of shape and size to enable said dispenser to be emplaced on either a hanging implement or a support; additionally comprising at least one of the following steps:
a. selecting said hanging implement from a group consisting of be a horizontal rod, a wire, a rope, a string, a hook, a shackle, a suction cup holder and any combination thereof; and
b. selecting said support from a group consisting of a leg, a stand and any combination thereof.

It is another object of the present invention to disclose the method as described above, additionally comprising a step of selecting a material of said dispenser from a group consisting of metal, plastic, silicone, cardboard, glass, paper and any combination thereof; additionally comprising at least one of the following steps:
a. selecting said metal comprising aluminum, iron and any combination thereof;
b. selecting a material of said plastic from a group consisting of rigid, semi-rigid, flexible and any combination thereof; and
c. selecting said plastic from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

It is another object of the present invention to disclose the method as described above, additionally comprising at least one of the following steps:
a. providing a member of a group consisting of said at least one adapter, said external compartment, said at least one inner compartment, and any combination thereof refillable; and
b. providing a member of a group consisting of said at least one adapter, said external compartment, said at least one inner compartment, and any combination thereof separable from said dispenser.

It is another object of the present invention to disclose the method as described above, additionally comprising steps of selecting a piped compartment from a group consisting of said external compartment, said at least one inner compartment and any combination thereof; and providing said piped compartment comprising a pipe with a lumen, said pipe providing fluid connection between said piped compartment and an exterior of said device; additionally comprising at least one of the following steps:
a. selecting said pipe from a group consisting of flexible, semi-flexible, rigid and any combination thereof;
b. selecting an external opening of said pipe from a group consisting of flush with the piped compartment, extending beyond a top of the piped compartment, and recessed into the piped compartment;
c. configuring said pipe to be either fixed relative to said lid or movable relative to said lid;
d. configuring an exit end of said pipe, when said lid is closed, to be flush with the piped compartment and, when said lid is open, said pipe extends beyond a top of the piped compartment;
e. configuring said pipe to be extendable beyond the top edge of the piped compartment; and
f. providing the pipe comprising a member of a group consisting of a lip pullable by a user, an accordion foldable section at said exit end, attached to the lid such that opening and closing the lid reversibly pulls the exit end of the pipe, having a flexible portion such that pulling or pushing on it extrudes the pipe, and any combination thereof.

It is another object of the present invention to disclose the method as described above, additionally comprising a step of providing the exit end of said pipe closeable by a conventional sealing closure selected from a group consisting of a cork, a clip-on fitting, a screw-on fitting, a friction-fit fitting, a slide-over fitting, a pinch fitting and any combination thereof.

It is another object of the present invention to disclose the method as described above, additionally comprising at least one of the following steps:
a. selecting said sealing closure from a group consisting of tamper-evident, child-proof and any combination thereof;
b. selecting said sealing closure comprising a member of a group consisting of a pour spout, a trigger, a sprayer cap, a pump, a thumb stop, a measuring attachment, a valve, an actuator and any combination thereof;
c. configuring at least one of said pipe and said sealing closure such that at least one of closure of the exit and pressure on the pipe forces contents of the pipe proximally; and
d. selecting said sealing closure from a group consisting of tamper-evident, child-proof and any combination thereof.

It is another object of the present invention to disclose the method as described above, additionally comprising a step of providing at least one wing configured by means of size and shape to prevent sliding between said adapter and a member of a group consisting of said roll of dry disposable wiping product, said external compartment, said inner compartment and any combination thereof.

It is another object of the present invention to disclose the method as described above, additionally comprising a step of selecting a configuration of said at least one wing from a group consisting of longitudinal corrugations, longitudinal ridges, transverse corrugations, transverse ridges, compressible pillows and any combination thereof.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates the prior art;

FIG. 2A-C schematically illustrates embodiments of a concentric dispenser;

FIG. 3A-C schematically illustrates an embodiment of an adapter or compartment that can change length;

FIG. 4 schematically illustrates an embodiment of a concentric dispenser with a central lumen;

FIGS. 5A-C and 6A-C schematically illustrate embodiments of dispensers;

FIG. 7A-B schematically illustrates embodiments of lids;

FIG. 8A-B schematically illustrates an embodiment of a stacked dispenser;

FIG. 9A-B schematically illustrates an embodiment of a dispenser with additional liquid dispenser;

FIG. 10 schematically illustrates an embodiment of a dispenser with a hanger;

FIG. 11A-E schematically illustrates embodiments of exemplary external side wings; and

FIG. 12A-C schematically illustrates embodiments of exemplary internal side wings.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for delivering a plurality of products from a single dispenser.

The term ‘product’ hereinafter refers to a quantity of a single item, typically as provided for use. For example, a stack of facial tissues for use in a dispenser is a product, as is a roll of kitchen towels. Two stacks of facial tissue, for use in two different dispensers, comprises two products. Similarly, a stack of facial tissue and a roll of kitchen paper is also two products. A collection of coins and bills (notes), intended to be stored together, would also constitute a single product. If, however, the coins were to be stored in one compartment and the bills were to be stored in a second compartment, then the coins would constitute one product and the bills a second product.

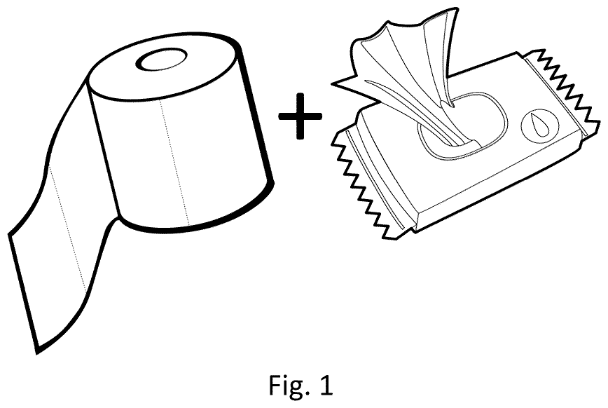

It is often desirable to use both dry disposable wiping product and wet (pre-moistened) disposable wiping product. Sometimes the same portion of the body is wiped with both, sometimes a dry disposable wiping product is used in one area and a wet disposable wiping product in another. In the prior art, this required two separate items, a package of dry disposable wiping product (typically a roll or stack of toilet paper, a roll of kitchen towels, or a pack of tissues) plus a dispenser carrying wet disposable wiping product (FIG. 1)

The dry disposable wiping product needs no special protection, except possibly for retaining cleanliness, but the wet disposable wiping product needs to be in resealable, impermeable packaging in order that it remain moist until use.

The present invention comprises a dispenser with both a dry disposable wiping product capability and a wet disposable wiping product capability.

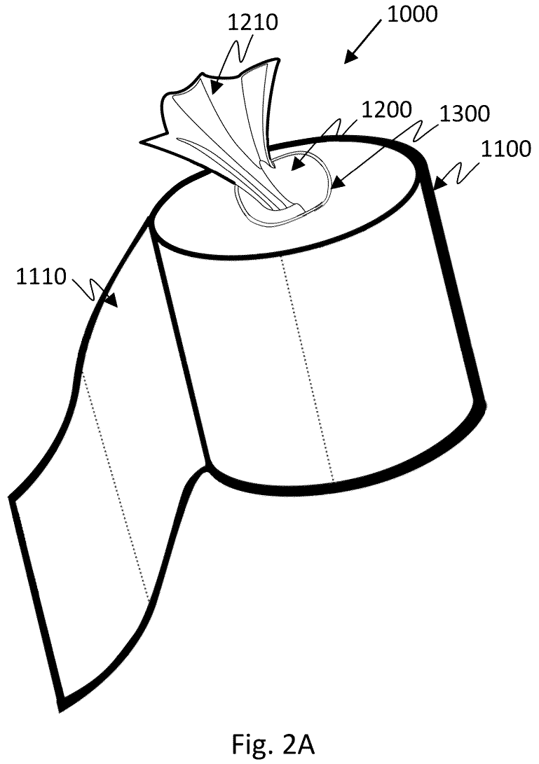

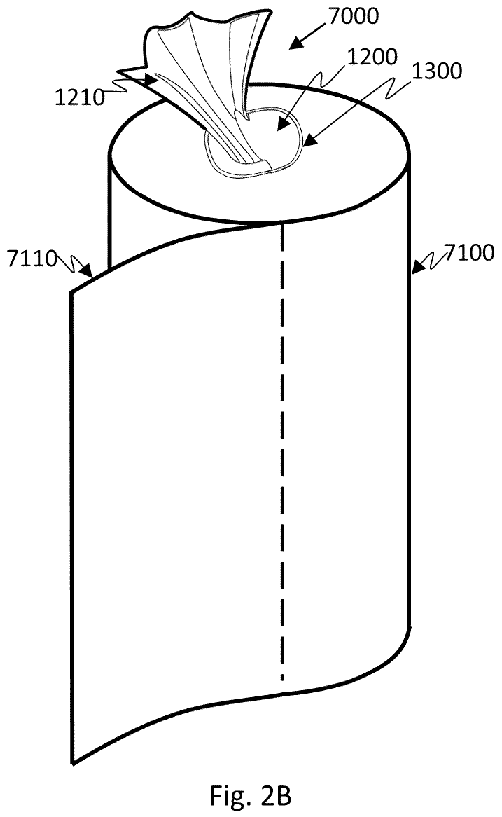

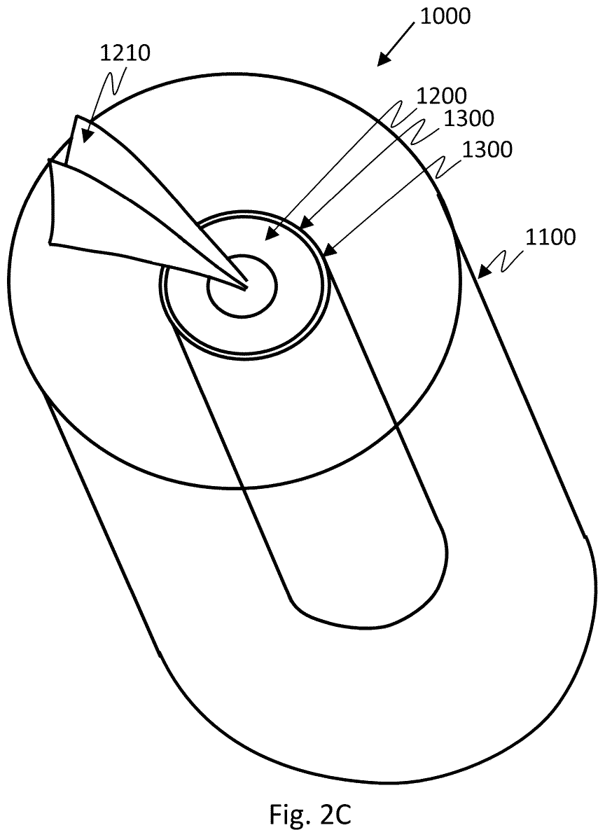

As schematically illustrated in the embodiments of FIG. 2A-C, the dispenser (1000) is shown in an exemplary loaded state, loaded with an external roll of dry disposable wiping product (1100) and with the inner compartment (1200) containing wet disposable wiping product (1210). The dispenser (1000) comprises an adapter (1300) which can be configured to fit either in the center of a roll (1100) of a dry disposable wiping product (1110) or to be attachable to an external compartment (shown hereinbelow). The dispenser (1000) comprises at least one inner compartment (1200). In the exemplary embodiment of FIG. 2, there is one inner compartment (1200), in this exemplary embodiment configured to contain wet disposable wiping product (1210). FIGS. 2A and 2B schematically illustrate an exterior view, while FIG. 2C schematically illustrates the central compartment within the roll (1100), the central compartment configured to contain pre-moistened wipes within the roll. In the exemplary embodiment of FIG. 2A, the roll is of toilet paper or another narrow product, whereas in the exemplary embodiment of FIG. 2B, the roll is of kitchen towels or other wide product. The outer roll is typically a dry disposable wiping product such as toilet paper, paper towels, bath tissue, roll towels, kitchen towels, kitchen rolls, cleaning cloths, reusable cloths, dish cloths, cleaning wipes or any other cleaning product commonly dispensed on rolls. The material of the roll product can be paper, woven cloth, non-woven cloth, or any other conventional material used for cleaning products dispensed on rolls.

The wet disposable wiping product can comprise any type of pre-moistened wipe. Non-limiting examples are wet wipes, toilet wipes, pre-moistened lens cleaning wipes, pre-moistened towelettes, pre-moistened makeup removal wipes, and any combination thereof. The material of the wet disposable wiping product can be paper, woven cloth, non-woven cloth, or any other conventional material used for cleansing products.

In some embodiments, an inner compartment, can be configured to contain any one of pre-moistened (wet) wipes, cleanser, lotion, dry disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, tampons, soap, shampoo, makeup remover, perfume, cologne, toilet water, antiperspirant, deodorant, makeup, cash, tokens or any other compartment filler as disclosed herein. The inner compartment can be of a shape and size to be inserted into the central hole of a roll of dry disposable wiping product, such as, but not limited to, the central hole in a roll of any one of the dry disposable wiping products disclosed herein; it can also be of a shape and size to be inserted in the central hole of an adapter configured to be a holder for dry disposable wiping product. This enables a user to create a combined dispenser with any desired roll of dry disposable wiping product. In some variants of this embodiment, the inner compartment is reversibly insertable so that a new compartment can be inserted into the hole when a current compartment is emptied. In some embodiments, the compartment is refillable; in some embodiments, the compartment is not refillable and must be replaced when empty.

In preferred embodiments, the inner compartment (2200) comprises a sealably reversibly closable lid (2210). In some embodiments, the compartment (2200) seals against a lid of the dispenser. In some embodiments, the compartment (2200) comprises a pipe for dispensing of the contents, as described hereinbelow. The pipe can be sealably closed by a lid.

The insertable compartment can be as long as the insertion hole, it can be longer than the insertion hole, it can be configured to project from the insertion hole while filling the insertion hole itself only partially, or it can be configured to fill the insertion hole only partially and not project beyond the boundaries of the insertion hole.

In some embodiments, the compartment can be permanently affixed within the insertion hole. The compartment can be as long as the insertion hole, it can be longer than the insertion hole, it can be configured to project from the insertion hole while filling the insertion hole itself only partially, or it can be configured to fill the insertion hole for only part of its length and not project beyond the boundaries of the insertion hole. In some embodiments, the exterior compartment is configured to contain wet disposable wiping product or other product as disclosed herein, while a compartment configured to contain dry disposable wiping product forms the interior compartment. The interior compartment, as above, can be insertable, reversibly insertable, or fixed. As above, the interior compartment can completely fill the length of the interior insertion hole, it can be longer than the insertion hole, it can be configured to project from the insertion hole while filling the insertion hole itself only partially, or it can be configured to fill the insertion hole for only part of its length and not project beyond the boundaries of the insertion hole.

In some embodiments, at least one of the adapter, an inner compartment and an outer compartment can be configured to allow change in length. For non-limiting example, a length-adjustable adapter could be fit into different length rolls of disposable wiping product, being usable, for non-limiting example, both for toilet paper and kitchen paper. A length-adjustable compartment could be adapted, for non-limiting example, for use with different size packs of wet disposable wiping product, different quantities of cleansing lotion, different size bottles of face cream, or a receptacle usable with both a long hairbrush and comb and a short razor and a small tube of shaving cream.

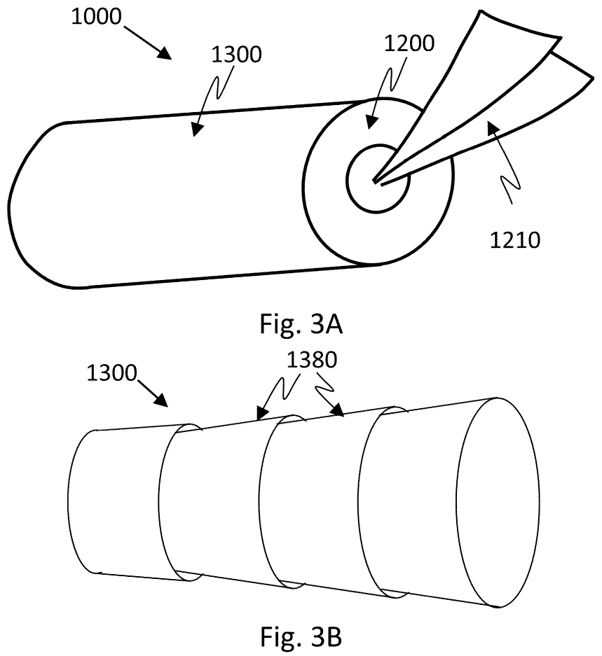

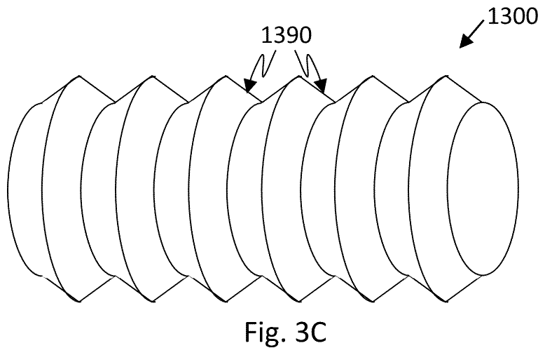

FIG. 3A schematically illustrates a device (1300) with a fixed-length adapter (1300) and an inner compartment (1200), the inner compartment (1200) containing, in this non-limiting example, wet disposable wiping product (1210). FIGS. 3B and 3C schematically illustrate non-limiting examples of extendable adapters. It should be noted that compartments can be extended and shortened by means of telescoping, accordion-type or other conventional means of changing compartment length, in the same manner as the adapter.

In the exemplary embodiment of FIG. 3B, the extensibility of the adapter (1300) is by means of telescoping sections (1280). In some embodiments, extensibility is provided by means of an accordion-pleated section (not shown). An extendable compartment or an extendable adapter can comprise a telescopic section, an accordion-pleated section, a removable section (not shown), and any combination thereof.

In the exemplary embodiment of FIG. 3B, the extensibility of the adapter (1300) is by means of telescoping sections (1280). In some embodiments, extensibility is provided by means of an accordion-pleated section (not shown). An extendable compartment or an extendable adapter can comprise a telescopic section, an accordion-pleated section, a removable section (not shown), and any combination thereof.

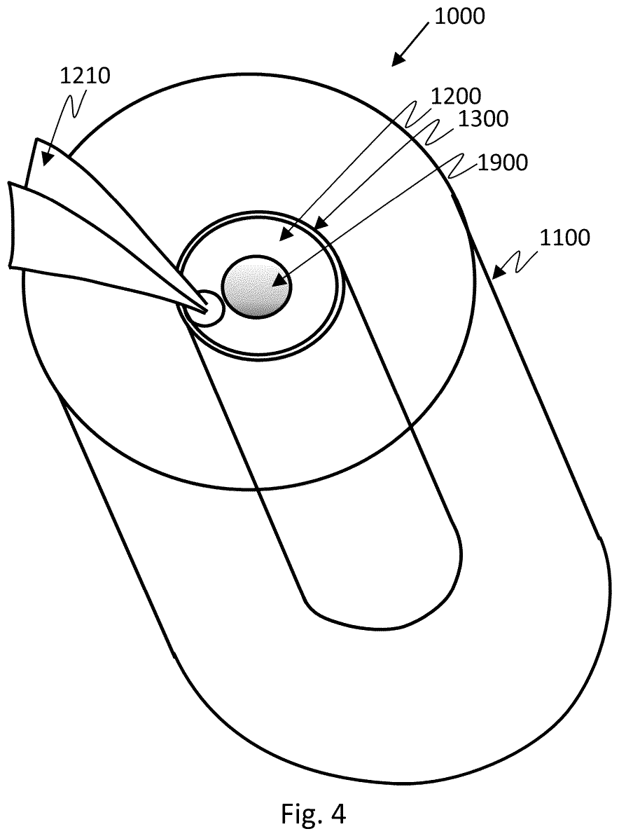

In some embodiments, as shown in FIG. 4, the interior compartment comprises a lumen (a channel) (1900) down at least part of its length, through which a hanging implement can be passed. The hanging implement can be any conventional hanging implement for non-limiting example, a horizontal rod, a wire, a rope, a string, a hook, a suction cup holder (held to a flat surface by suction), a shackle and any combination thereof.

As schematically illustrated in the embodiment of FIG. 4 the dispenser (1000) is shown in an exemplary loaded state, with an external roll of dry disposable wiping product (1100) securely held by the adapter (1300) and with the inner compartment (1200) containing wet disposable wiping product (1210).

In some embodiments, the interior compartment is provided without contents so that a user can add desired contents at the time of use. This eases long-term storage, in that there is no danger of pre-moistened wipes losing moistening during storage, as they are added at point of use. It also enables a user to select a desired filler for the interior compartment, such as a brand, or a type of filling material (lotion, wiping product, etc.).

Non-limiting examples of compartment fillers comprise dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, a cleansing agent, hand lotion, body lotion, makeup, tampons, soap, soap pads, shampoo, makeup remover, perfume, cologne, toilet water, antiperspirant, deodorant, cash, and tokens

In some embodiments, an exterior roll is attachable to a roll holder. This enables a user to select a desired exterior roll or exterior compartment for attachment to the dispenser.

A compartment can have any of the configurations disclosed herein; (a) it can be permanently attached to the outside roll or roll holder, insertably attached to the outside roll or roll holder, removably attached to the outside roll or roll holder, and reversibly (insertably and removably) attached to the outside roll or roll holder; (b) it can be the entire length of the lumen of the roll or roll holder, only part of the length of the lumen of the roll or roll holder or longer than the lumen of the roll or roll holder; (c) it can protrude from the lumen of the roll or roll holder, be flush with one or more ends of the lumen of the roll or roll holder, or protrude from the lumen of the roll or roll holder; (d) it can fill the entire diameter of the lumen of the roll or roll holder or it can have a compartment lumen to allow passage of a hanging implement, and any combination of (a), (b), (c) and (d).

The dispenser can be provided as a roll holder and one or more empty compartments, thus enabling a user to select freely both the exterior wipes and the interior wipes.

The roll holder and compartment(s) can be provided as separate items, or can be provided as a unit. The roll holder and compartments can be integral or can be separable. Compartments can be provided filled or empty.

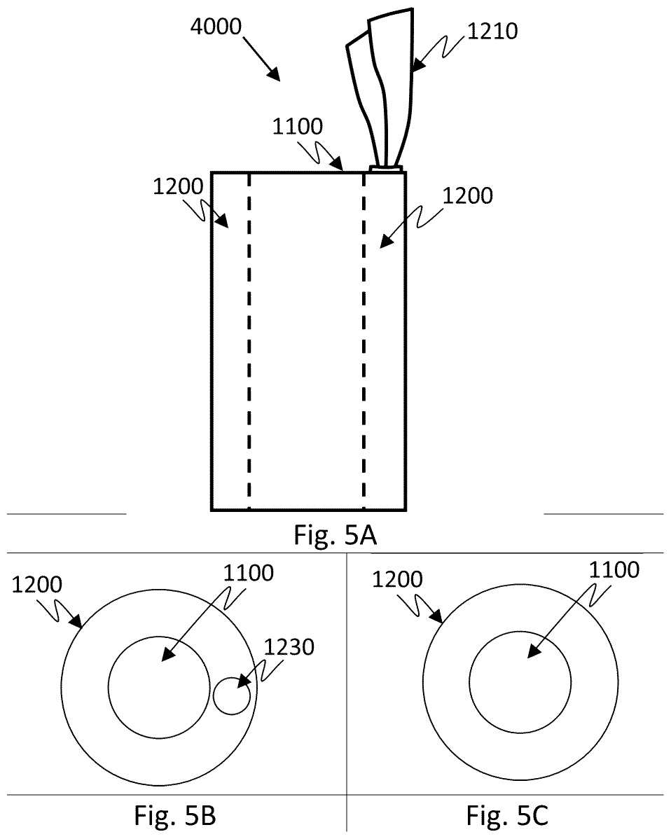

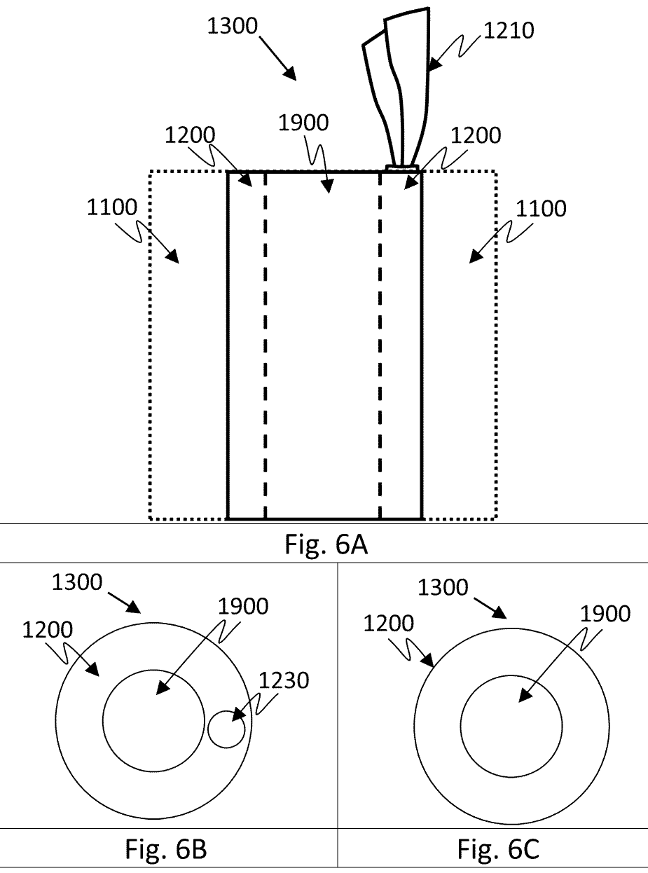

In the embodiment of the dispenser (4000) schematically illustrated in FIG. 5A-C, the dry wipe roll (1100) is on the interior and the pre-moistened wipe compartment (1200) is on the outside. FIG. 5A shows the dispenser (4000) from the side, FIG. 5B shows the dispenser (4000) from the top; the opening (1230) to allow passage of the pre-moistened wipes can be seen, and FIG. 5C shows the dispenser (4000) from the bottom.

In the embodiment of the adapter (1300) schematically illustrated in FIG. 6A-C, the adapter comprises a compartment (1200) with a central lumen (1900). In FIG. 6A, the location of a dry wipe roll (1100) is schematically indicated by the dotted line. In the exemplary embodiment shown, the compartment (1200) contains wet disposable wiping product (1210). The dashed vertical lines in FIG. 6A indicate the extent of the lumen (1900). FIG. 6B schematically illustrates the adapter (1300) from the top, showing the lumen (1900) and the opening (1230) to allow passage of the pre-moistened wipes, and FIG. 6C shows the adapter (1300) from the bottom. The lumen (1900) can be used to hold a packet, package or bottle; more typically, a holder, as disclosed hereinbelow, can pass through the lumen (1900), to enable the adapter (1300) to be supported from above; or a stand or legs can be inserted through the lumen (1900) to support it from below.

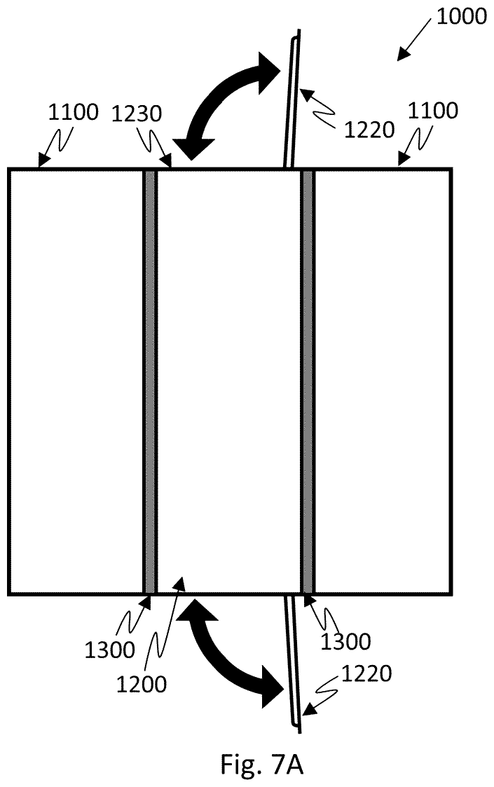

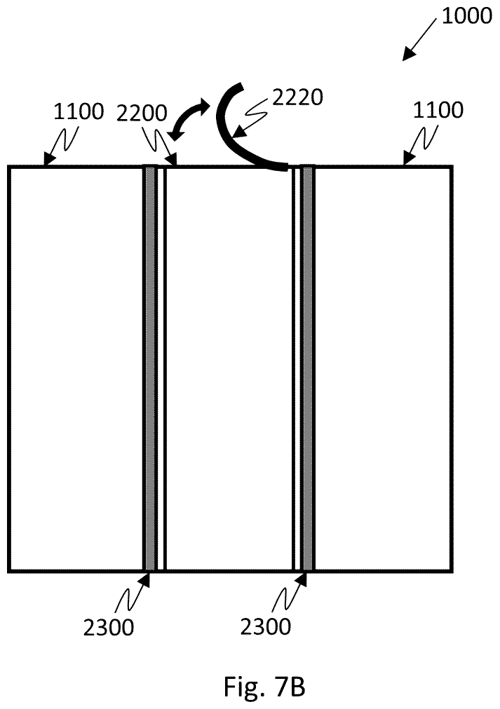

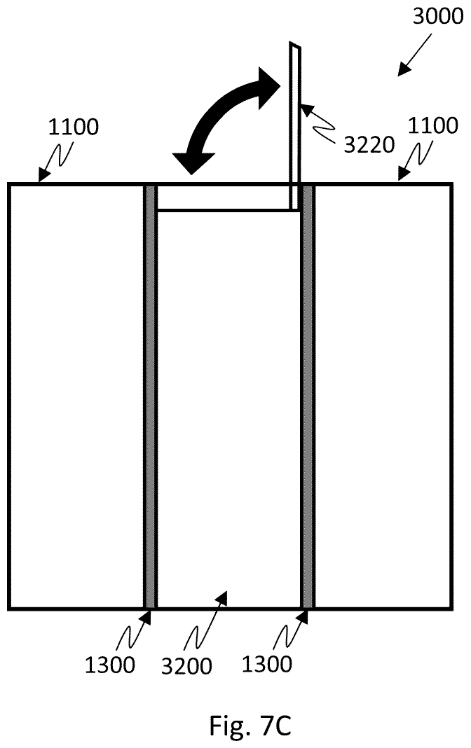

FIG. 7A-C schematically illustrates embodiments of closures for an interior compartment of the dispenser (1000). In FIG. 7A-C, the exterior roll (1100) is shown. In FIG. 7A, the interior compartment (1200) is fixedly attached to the adapter (1300), while in FIG. 7B, the interior compartment (1200) is insertably (or reversibly insertably) attached to the adapter (2300). FIG. 7A schematically illustrates a dispenser with a closure at both ends, while FIGS. 7B and 7C schematically illustrate dispensers with closures at only one end.

A dispenser with closures at both ends will typically comprise two compartments, each held closed by one of the lids. Typically, the contents of the compartments will differ. For non-limiting example, one compartment could contain wet disposable wiping product, while the other contains hand lotion. In another example, one compartment contains wet disposable wiping product, while the other contains money. Non-limiting examples of compartment contents are dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, a cleansing agent, hand lotion, body lotion, makeup, tampons, soap, soap pads, shampoo, makeup remover, perfume, cologne, toilet water, antiperspirant, deodorant, cash, and tokens.

The bag can be any flexible container, such as, for non-limiting example, a trash bag for disposal of waste, a grocery bag for a purchase, a protective bag for a liquid container that has developed a leak, and an impermeable bag for animal waste. A plurality of bags can comprise a roll of bags, a stack of bags, a plurality of individual bags, and any combination thereof.

The receptacle can be configured to hold items ready for use. For non-limiting example, the receptacle can be configured to contain a tube of toothpaste, a toothbrush and dental floss and perhaps a small container of water, so that the items are convenient to hand during travel. In another non-limiting example, the receptacle can be configured to contain a razor blade and shaving cream. In yet another non-limiting example, the receptacle can contain a bottle of drinkable liquid and cutlery. In some embodiments, the device can comprise fold-out legs to stabilize it to reduce the probability that the device tips or tilts when the receptacle is being used.

In FIG. 7A, the closure is a rigid, semi-rigid, flexible and any combination thereof lid (1220) that is rotatable (double-headed arrow) about one end, the end being hingedly attached to the cap (the exterior end) of the interior compartment (1200). The lid (1220) is of conventional construction and can be flat or can have one or more ridges. When closed, the lid (1220) provides a sealing closure of any conventional type, for non-limiting example, a snap-fit into a matching opening in the cap of the compartment (1200) via one or more grooves in the edge of the lid (1220), a snap-fit into a matching opening in the cap of the compartment (1200) via one or more grooves in the edge of the hole in the end of the interior compartment (1200), a friction fit between one or more ridges on the edge of the lid (1220) and a matching opening in the cap, a friction fit between one or more ridges on the edge of the lid (1220) and one or more ridges on the edge of a matching opening in the cap, a clip joining the lid (1220) to the cap, a hook joining the lid (1220) to the cap, a shackle joining the lid (1220) to the cap, adhesive around an edge either of the cap or the lid where the lid and cap overlap, and any combination thereof.

In FIG. 7B, the closure is a flexible lid (2220) that is rotatable (double-headed arrow) about an end attached to the interior compartment (2200). The flexible lid (2220) is of conventional construction and can be flat or can have one or more ridges. When closed, the flexible lid (2220) provides a sealing closure of any conventional type, typically by means of adhesive around an edge either of the interior compartment (2200) or on the lid where the lid and cap overlap, so that the adhesive adheres the edge of the flexible lid (2220) to the edge of the interior compartment (2200). Any other conventional means of sealing a flexible lid to an interior compartment (2200) can be used. Non-limiting examples are a snap-fit into a matching opening in the cap of the compartment (2200) via one or more grooves in the edge of the lid (2220), a snap-fit into a matching opening in the cap of the compartment (2200) via one or more grooves in the edge of the hole in the end of the interior compartment (2200), a friction fit between one or more ridges on the edge of the lid (2220) and a matching opening in the cap, a friction fit between one or more ridges on the edge of the lid (2220) and one or more ridges on the edge of a matching opening in the cap, a clip joining the lid (2220) to the cap, a hook joining the lid (2220) to the cap, a shackle joining the lid (2220) to the cap, and any combination thereof.

In the embodiment of FIG. 7C, the dispenser (3000) has a lid (3220) recessed into the interior compartment (3200). In the exemplary embodiment shown, the lid (3220) is rigid or semi-rigid, although a recessed lid can also be flexible. In the exemplary embodiment shown, the lid extends all the way across the top edge of the interior compartment (3200). In other variants of a recessed lid, the interior compartment (3200) can have an opening, as described above for FIGS. 5A-B. Any method of sealing the lid (3220) against the interior compartment (3200), as disclosed above, can be used for a recessed lid (3220). The outer side of a recessed lid (3220) can be proud of the top of the interior compartment (3200), flush with the top of the interior compartment (3200), or lower than the top of the interior compartment (3200).

In various embodiments, a lid can be hinged (as in the embodiment of FIG. 7A), can have adhesive around an edge, can screw on, can clip on, can slide on, can have a friction fit, and any combination thereof. A lid can comprise metal, plastic, silicone, cardboard, glass, paper, cork and any combination thereof. A lid can be tamper-evident, child-proof and any combination thereof. A lid can be a dispensing lid, a spray lid and any combination thereof. A dispensing lid can comprise a pour spout, a trigger, a sprayer cap, a pump, a measuring attachment, a sifting device, a valve, an actuator and any combination thereof.

A lid can be permanently attached to a dispenser or a compartment or it can be releasable from the dispenser or compartment. The means of attachment can be any conventional means of attaching a lid to a container, for non-limiting example, a hinge, a ligament attached at one end to the lid and at the other to the dispenser, a ligament attached at one end to the lid and at the other to a compartment, a spring, and any combination thereof. The ligament is preferably flexible and can comprise metal, plastic, silicone or cloth (e.g., a string). The ligament can be integral to the lid or attached to the lid,

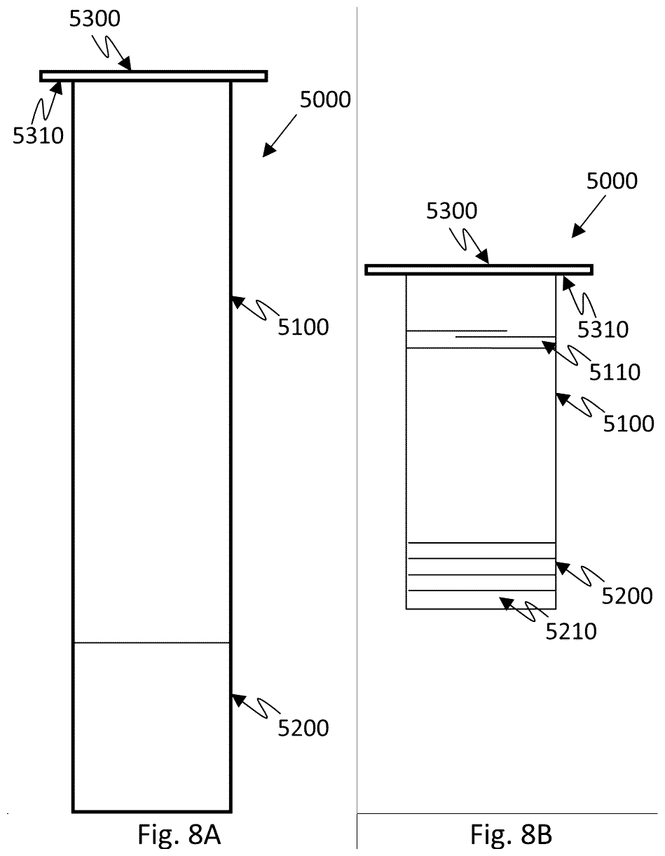

FIG. 8A-B schematically illustrates a dispenser (5000) in which the holders for the two types of wipes (5100, 5200) are arranged vertically, one above the other, instead of concentrically. The embodiment of FIG. 8A is intended for a narrower paper, such as toilet paper, while the embodiment of FIG. 8B is intended for a wider paper, such as kitchen towels.

In the exemplary embodiments shown, the dry disposable wiping product is on a roll and the pre-moistened wipes are stacked. Any conventional form of stacking can be used, as long as removal of one wipe prepares a next wipe for removal. In the exemplary embodiments shown, the dry wipe holder (5100) is above the pre-moistened wipe holder (5200). The dry disposable wiping product (not shown) can be removed from the dry disposable wiping product holder (5100) by means of a reversibly openable shutter (5110). The pre-moistened wipes (5210) can be removed from the pre-moistened wipe holder (5200) either by means of a reversibly openable shutter (not shown) or by means of a closable opening, as disclosed above.

In some variants, the pre-moistened wipes (5210) are removed by means of a shutter or opening, as disclosed above, in the base of the dispenser (5000).

In some variants, the dispenser (5000) can be suspended by means of the flange (5310) of the top cover (5300), which extends outward beyond the edges of the holders (5100, 5200). In some variants, the dispenser (5000) rests on its bottom.

In some embodiments, the dispenser (5000) additionally comprises a holder for a third cleansing means (not shown), such as the liquid or semi-liquid cleanser disclosed above. The holder for a third cleansing means can be at the top of the stack, in the central portion of the stack between the dry wipe holder (5100) and the pre-moistened wipe holder (5200), or at the bottom of the stack.

In some embodiments, the pre-moistened wipe holder (5200) is above the dry wipe holder (5100).

In some embodiments, at least two of the holders are separable. In some embodiments, at least two of the holders are unitary. In some embodiments, at least one holder is refillable.

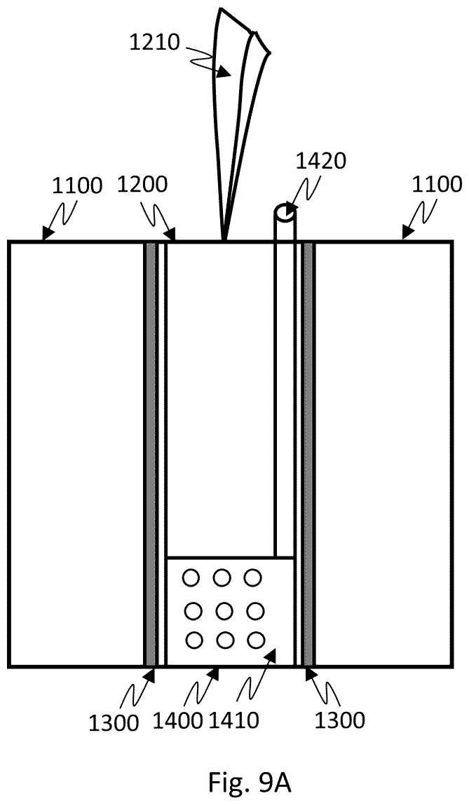

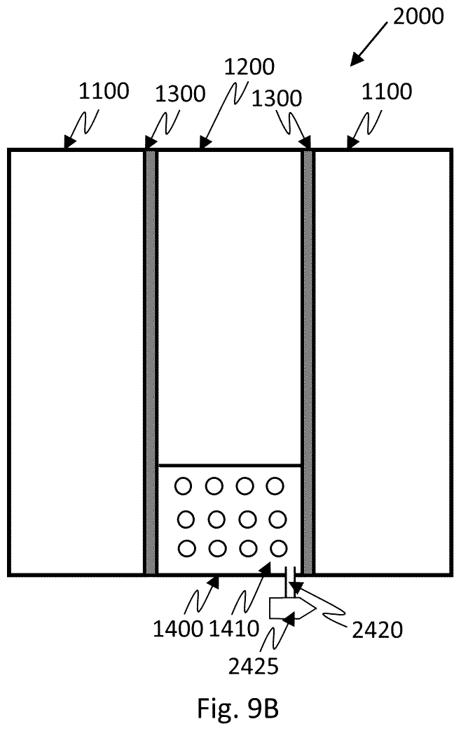

FIG. 9A-B schematically illustrates an embodiment of a dispenser (2000) with three types of cleansing means. In this embodiment, the outer roll (1100) comprises dry disposable wiping product, the first inner compartment (1200) comprises pre-moistened wipes (1210), and the second inner compartment (1400) comprises a liquid or semi-liquid cleanser (1410) such as, but not limited to, alcohol, an alcohol gel, an antiseptic, hand cleanser and any combination thereof.

In the embodiment of FIG. 9A, the first interior compartment (1200) and the second interior compartment (1400) are removably insertable into the adapter (here, a roll holder) (1300). In the embodiment of FIG. 7B, the first interior compartment (1200) and the second interior compartment (1400) are fixedly attached to the adapter (1300)

In the embodiment of FIG. 9A, the cleanser (1410) exits (2425) the second inner compartment (1400) via the same end of the dispenser (2000) as the pre-moistened wipes (1210).

In some embodiments, the dispenser with more than one compartment is unitary. In other embodiments, at least two of the compartments comprise a unit. In preferred embodiments, the adapter is separable from the compartments and the compartments are separable from each other. The contents of the compartments can be any of the products disclosed herein.

The fluid connection (1420) between the second inner compartment (1400) and the exit, as disclosed hereinabove, can be of any conventional type, most typically a pipe, flexible, semi-flexible, rigid and any combination thereof, with a lumen. The pipe (1420) opening (2425) can be flush with the second inner compartment (1400), extend beyond the top of second inner compartment (1400), or (less preferred) be recessed into the second inner compartment (1400). The pipe (1420) can be fixed relative to the second inner compartment (1400) or movable relative to the second inner compartment (1400). In some embodiments, the pipe (1420), when closed, is flush with the second inner compartment (1400) and, when open, extends beyond the top of the second inner compartment (1400). To extend the pipe beyond the top edge of the second inner compartment (1400), the pipe can comprise a lip pullable by a user, can comprise an accordion foldable section at its exit end, can be attached to the lid such that opening and closing the lid reversibly pulls the end of the pipe, the second inner compartment (1400) can comprise a flexible portion such that pushing on it extrudes the pipe, and any other conventional means of extending an retracting an end of the pipe.

The external end of the exit can be closed by any conventional sealing closure such as, but not limited to, a cork, a clip-on fitting, a screw-on fitting, a friction-fit fitting, a slide-over fitting, a pinch fitting and any combination thereof.

A pipe closure can be tamper-evident, child-proof and any combination thereof. A pipe can have a dispenser or a spray attachment. A pipe closure can comprise a pour spout, a trigger, a sprayer cap, a pump, a measuring attachment, a valve, a thumb stop, an actuator and any combination thereof.

In preferred embodiments, the inner compartment (1400) is refillable. In other embodiments, the inner compartment (1400) is not refillable and must be discarded after emptying.

In some embodiments, the pipe is configured such that at least one of closure of the exit and pressure on the pipe forces contents of the compartment which are in the pipe proximally, back into the compartment and out of the pipe, thus preventing dripping from the pipe.

In some embodiments, the closure comprises a thumb stop which, when pressed into the closed position, both closes the pipe and forces the contents of the inner compartment proximally toward the interior of the inner compartment.

In the exemplary embodiment of FIG. 8B, the cleanser (1410) exits (2425) the second inner compartment (1400) via the opposite end of the dispenser (2000) from the first inner compartment (1200). The fluid connection (2420) between the second inner compartment (1400) and the exit can be of any conventional type, most typically a pipe, flexible, semi-flexible, rigid and any combination thereof, with a lumen. The pipe (2420) opening (2425) can be flush with the second inner compartment (1400), extend beyond the top of the second inner compartment (1400), or (less preferred) be recessed into the second inner compartment (1400). The pipe (2420) can be fixed relative to the second inner compartment (1400) or movable relative to the second inner compartment (1400). In some embodiments, the pipe (2420), when closed, is flush with the second inner compartment (1400) and, when open, extends beyond the top of the second inner compartment (1400). To extend the pipe beyond the top edge of the second inner compartment (1400), the pipe can comprise a lip pullable by a user, can comprise an accordion foldable section at its exit end, can be attached to the lid such that opening and closing the lid reversibly pulls the end of the pipe, the second inner compartment (1400) can comprise a flexible portion such that pushing on it extrudes the pipe, and any other conventional means of extending an retracting an end of the pipe.

The external end of the exit can be closed by any conventional sealing closure such as, but not limited to, a cork, a clip-on fitting, a screw-on fitting, a friction-fit fitting, a slide-over fitting, a pinch fitting and any combination thereof.

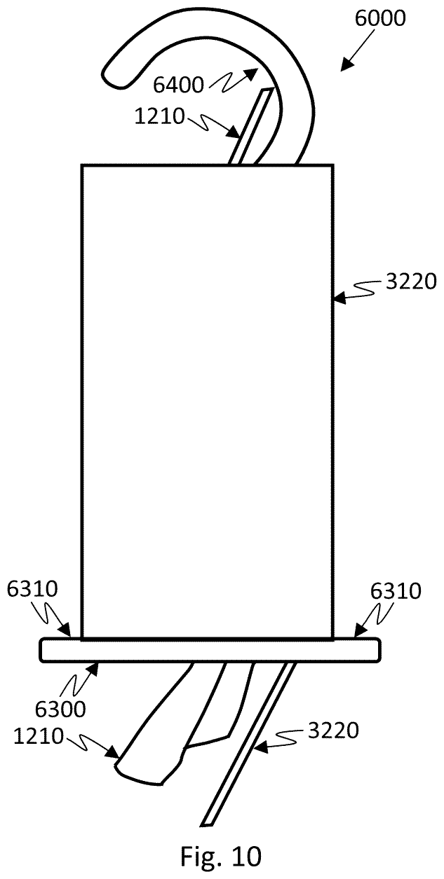

The embodiment of FIG. 10 schematically illustrates an embodiment similar to that of FIG. 9, where the two compartments are one above the other inside the dispenser (6000). In this exemplary embodiment, the lower compartment is configured to contain wet disposable wiping product and the upper compartment configured to contain another product, for non-limiting example, dry disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, a cleansing agent, hand lotion, body lotion, makeup, tampons, soap, shampoo, makeup remover, perfume, cologne, toilet water, antiperspirant, deodorant, cash, tokens or any other product as disclosed herein.

In the embodiment of FIG. 10, the dispenser (6000) can be suspended by means of a hanging implement. In the exemplary embodiment shown, the hanging implement is a hanging hook (6400). The hanging implement can be a horizontal rod, a wire, a rope, a string, a hook, a shackle, a suction cup holder (held to a flat surface by suction) and any combination thereof.

The hanging implement can be of any conventional rigid or semi-rigid material, such as, but not limited to, metal or plastic. In some variants, the hanging implement can be folded down to the holder body for more compact storage. In some variants, the hanging implement can be folded along a longitudinal axis, both to reduce storage volume and to prevent an end of the hanging implement from catching on other items during storage. The means of folding can be any conventional folding means, such as, but not limited to, at least one hinge, at least one flexible section and any combination thereof.

The hanging implement can be removable from the dispenser, it can be can be removable from a compartment, it can be permanently attached to the dispenser, and it can be can be permanently attached to a compartment.

A dispenser can comprise any conventional material, such as, but not limited to, metal (typically aluminum or steel), a rigid plastic, a semi-rigid plastic, a flexible plastic and any combination thereof. The plastic can comprise nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

Lids for refillable holders can comprise screw-on fittings, clip-on fittings or any other type of reversibly closable and sealable fitting.

In some embodiments, at least a portion of at least one holder can be of a flexible plastic, allowing the holder to be at least partially collapsed for storage.

Dry disposable wiping product can comprise a roll or stack of toilet paper, a roll of kitchen towels, a pack of tissues, surface wipes, hand cleaning wipes and any combination thereof.

Pre-moistened wipes can comprise baby wipes, deodorant wipes, disinfectant wipes, personal hygiene wipes, cleansing pads, industrial wipes, pain relief pads, eye cleansing pads, ear cleansing pads, dental cleansing pads, alcohol wipes, sporicidal wipes and any combination thereof.

Lids can be flat or curved.

The outside rim can provide a recess so that wipes do not protrude too far.

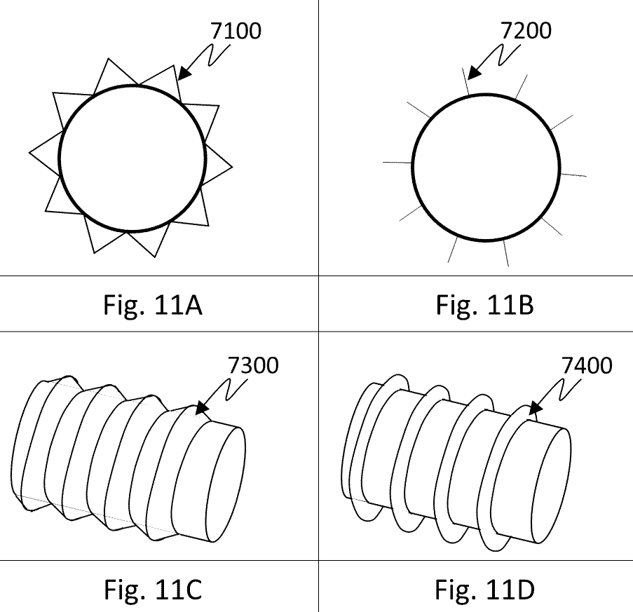

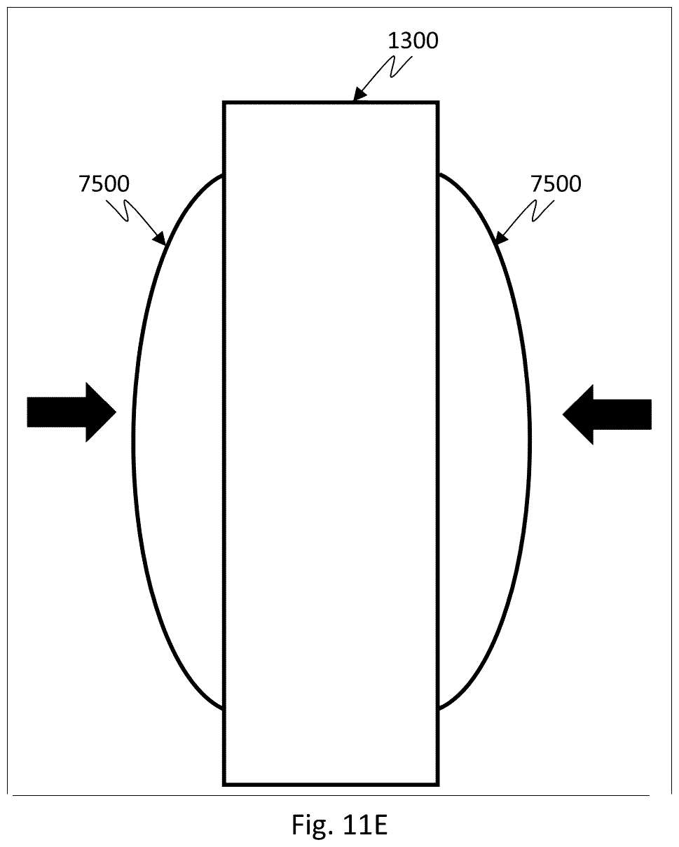

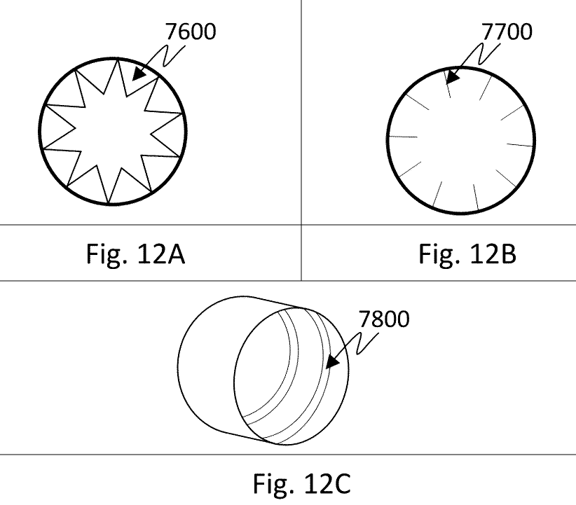

The dispenser dry wipe holder can further comprise external flexible wings so that external dry wipe rolls with different diameter interior holes can be held snugly by the adapter (FIG. 11A-E); internal flexible side wings so that inner compartments, rolls or packets of different diameters can be held snugly by the adapter (FIG. 12A-C); and any combination thereof.

As schematically illustrated in FIG. 11A-D, flexible external side wings can be in the form of longitudinal corrugations (FIG. 11A, 7100), longitudinal ridges (FIG. 11B, 7200), transverse corrugations (FIG. 11C, 7300), transverse ridges (FIG. 11D, 7400), compressible pillows (FIG. 11E, 7500) or any other conventional shape configured to prevent unwanted sliding between two cylinders.

In FIG. 11E, the flexible external wings are in the form of rounded pillows (7500); in the embodiment shown, there is one on each side of the adapter (1300). When the external roll or compartment is lowered onto the adapter (1300), the pillows (7500) compress (black arrows). The pillows are configured to be resilient so that they will expand to at least partially fill the space between the adapter (1300) and the external roll (not shown). There can be a single pillow (7500) or a plurality of pillows (7500). In some embodiments, the single pillow (7500) extends entirely around the adapter (1300), in other embodiments, the single pillow (7500) extends only partway around the adapter (1300). A pillow (7500) can extend the entire length of the adapter (1300) or along only part of the length of the adapter (1300). The pillow can comprise a compressible, resilient material, it can comprise a flexible material, it can contain gas, and any combination thereof. Typically, a pillow (7500) which contains gas will have a flexible cover; the cover need not be resilient.

As schematically illustrated in FIG. 12A-C, flexible internal side wings can be in the form of longitudinal corrugations (FIG. 12A, 7600), longitudinal ridges (FIG. 11B, 7700), transverse corrugations (not shown), transverse ridges (FIG. 11C, 7800), compressible pillows (not shown) or any other conventional shape configured to prevent unwanted sliding between two cylinders.

In any of the embodiments disclosed above except that of FIG. 5, which discloses a dry wipe roll (1100) on the interior and a pre-moistened wipe compartment (1200) on the outside, the dispenser is configured to firmly support an outer roll. The outer roll can be any dry wipe as disclosed above.

In some embodiments with dry wipe roll on the outside (outer roll), the dispenser comprises a permanently-attached outer roll. In preferred embodiments, the outer roll is removably emplaceable on the dispenser. In such preferred embodiments, any outer roll with a central hole of an appropriate diameter can be emplaced on the dispenser.

In some embodiments, the longitudinally disposed compartment(s) (one at each end) are annular, with a lumen in the center. In some variants of these embodiments, the lumen is configured to accept a rod-like toilet roll holder, as disclosed above. In other variants of theses embodiments, the lumen is configured to contain one or two further longitudinally-disposed compartments.

In all of the dispensers disclosed herein, in some variants of embodiments of dispensers, at least one of the compartments is permanently attached to the dispenser. In some variants of embodiments of dispensers, at least one of the compartments is reversibly attachable (it can be emplaced and it can be removed) to at least one of a group consisting of the dispenser, another compartment and any combination thereof.

In all of the dispensers disclosed herein, in some variants of embodiments of compartments and dispensers with compartments, at least one of the compartments is refillable. In some variants of embodiments of dispensers, at least one of the compartments is not refillable and must be disposed of after emptying.

In some embodiments, the dispenser and at least one compartment is supplyable as a single unit. In some variants of these embodiments, at least one compartment is supplied filled.

In some embodiments, the dispenser is supplyable as a single unit, any compartment attachable to or insertable in the dispenser being supplyable separately.

In some variants of embodiments of compartments, the compartment is supplyable filled. In some variants of embodiments of compartments, the compartment is supplyable empty.

In preferred embodiments of compartments, the compartment is refillable. In less-preferred embodiments of compartments, the compartment must be discarded after emptying.

1.-50. (canceled)

51. A dispenser for containing a plurality of products characterized by:
at least one adapter, said at least one adapter configured to securely hold at least one first product; said at least one first product being holdable directly to said adapter; and
at least one inner compartment configured to contain at least one second product, said at least one inner compartment at least partially within a lumen of said adapter;
said at least one adapter and said at least one inner compartment being unitary or separable wherein said at least one inner compartment comprises a reversibly closable lid, said closable lid being sealably closable; at least one of the following being true:
a. a closure of said closable lid is selected from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and in said at least one inner compartment overlap, and any combination thereof;
b. said closable lid is flexible, semi-rigid, rigid, and any combination thereof;
c. a location of an external side of said closable lid is selected from a group consisting of proud of said at least one inner compartment, flush with said at least one inner compartment and recessed below an external side of said at least one inner compartment;
d. said closable lid comprises a material selected from a group consisting of metal, plastic, silicone, cardboard, glass, paper, cork and any combination thereof;
e. said closable lid is selected from a group consisting of tamper-evident, child-proof and any combination thereof;
f. said closable lid is selected from a group consisting of a dispensing lid, a spray lid and any combination thereof; and
g. said dispensing lid comprises a member of a group consisting of a pour spout, a trigger, a sprayer cap, a pump, a measuring attachment, a sifting device, a valve, an actuator and any combination thereof.

52. The dispenser of claim 51, wherein said at least one first product is selected from a group consisting of dry disposable wiping product, wet disposable wiping product, and any combination thereof.

53. The dispenser of claim 51, wherein said at least one second product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
a. said cleansing agent is selected from a group consisting of a liquid, a semi-liquid and a gel; and
b. said cleansing agent is selected from a group consisting of alcohol, an alcohol gel, an antiseptic, hand cleanser and any combination thereof.

54. The dispenser of claim 51, additionally comprising a hollow lumen configured by means of shape and size to enable said dispenser to be emplaced on either a hanging implement or a support; at least one of the following being true:
a. said hanging implement is selected from a group consisting of be a horizontal rod, a wire, a rope, a string, a hook, a shackle, a suction cup holder and any combination thereof; and
b. said support is selected from a group consisting of a stand, a leg and any combination thereof.

55. The dispenser of claim 51, wherein a material of said dispenser is selected from a group consisting of metal, plastic, silicone, cardboard, glass, paper and any combination thereof.

56. The dispenser of claim 55, wherein at least one of the following is true:
a. said metal comprises aluminum, iron and any combination thereof;
b. a material of said plastic is selected from a group consisting of rigid, semi-rigid, flexible and any combination thereof; and
c. said plastic is selected from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

57. The dispenser of claim 51, wherein at least one of the following is true:
a. a member of a group consisting of said at least one adapter, said at least one compartment and any combination thereof is refillable; and
b. a member of a group consisting of said at least one adapter, said at least one compartment and any combination thereof is separable from said dispenser.

58. The dispenser of claim 51, additionally comprising a pipe with a lumen, said pipe providing fluid connection between said at least one inner compartment and an exterior of said device.

59. The dispenser of claim 58, wherein at least one of the following is true:
a. said pipe is selected from a group consisting of flexible, semi-flexible, rigid and any combination thereof;
b. an external opening of said pipe is selected from a group consisting of flush with the at least one inner compartment, extend beyond the top of the at least one inner compartment, and be recessed into the at least one inner compartment;
c. said pipe is either fixed relative to the at least one inner compartment or movable relative to the at least one inner compartment;
d. said pipe, when said lid is closed, is flush with the at least one inner compartment and, when said lid is open, extends beyond the top of the at least one inner compartment;
e. said pipe is extendable beyond the top edge of the at least one inner compartment; and
f. the pipe comprises a member of a group consisting of a lip pullable by a user, an accordion foldable section at its exit end, be attached to the lid such that opening and closing the lid reversibly pulls the end of the pipe, a flexible portion such that pushing on it extrudes the pipe, and any combination thereof.

60. The dispenser of claim 59, wherein an exit end of said pipe is closeable by a conventional sealing closure selected from a group consisting of a cork, a clip-on fitting, a screw-on fitting, a friction-fit fitting, a slide-over fitting, a pinch fitting and any combination thereof.

61. The dispenser of claim 60, wherein at least one of the following is true:
a. said sealing closure is selected from a group consisting of tamper-evident, childproof and any combination thereof;
b. said sealing closure is selected from a member of a group consisting of a pour spout, a trigger, a sprayer cap, a pump, a thumb stop a measuring attachment, a valve, an actuator and any combination thereof;
c. at least one of said pipe and said sealing closure is configured such that at least one of closure of the exit and pressure on the pipe forces contents of the pipe proximally; and
d. said sealing closure is selected from a group consisting of tamper-evident, childproof and any combination thereof.

62. The dispenser of claim 51, additionally comprising at least one wing configured by means of size and shape to prevent sliding between said adapter and a member of a group consisting of said first product, said inner compartment and any combination thereof said at least one wing having a configuration selected from a group consisting of longitudinal corrugations, longitudinal ridges, transverse corrugations, transverse ridges, compressible pillows and any combination thereof.

63. A dispenser for containing a plurality of products comprising:
at least one adapter, said at least one adapter configured to securely hold at least one first product; said at least one first product being holdable in a manner selected from a group consisting of directly to said adapter, in an external compartment and any combination thereof; and
at least one inner compartment configured to contain at least one second product, said at least one inner compartment at least partially within a lumen of said adapter;
wherein a member of a group consisting of said external compartment, said at least one inner compartment and any combination thereof comprises a reversibly closable lid, said closable lid being sealably closable; at least one of the following being true:
a. a closure of said closable lid is selected from a group consisting of a snap-fit into a matching opening in the at least one inner compartment via one or more grooves in the edge of the closable lid, a snap-fit into a matching opening in the at least one inner compartment via one or more grooves in the edge of the hole in the end of the at least one inner compartment, a friction fit between one or more ridges on the edge of said closable lid and a matching opening in said at least one inner compartment, a friction fit between one or more ridges on the edge of said closable lid and one or more ridges on the edge of a matching opening in said at least one inner compartment, a clip joining the closable lid to said at least one inner compartment, a hook joining the closable lid to said at least one inner compartment, a shackle joining the closable lid to said at least one inner compartment, adhesive around an edge either of said at least one inner compartment or the closable lid where the closable lid and in said at least one inner compartment overlap, and any combination thereof;
b. said closable lid is flexible, semi-rigid, rigid, and any combination thereof;
c. a location of an external side of said closable lid is selected from a group consisting of proud of said at least one inner compartment, flush with said at least one inner compartment and recessed below an external side of said at least one inner compartment;
d. said closable lid comprises a material selected from a group consisting of metal, plastic, silicone, cardboard, glass, paper, cork and any combination thereof;
e. said closable lid is selected from a group consisting of tamper-evident, child-proof and any combination thereof;
f. said closable lid is selected from a group consisting of a dispensing lid, a spray lid and any combination thereof; and
g. said dispensing lid comprises a member of a group consisting of a pour spout, a trigger, a sprayer cap, a pump, a measuring attachment, a sifting device, a valve, an actuator and any combination thereof.

64. The dispenser of claim 63, wherein said at least one first product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
h. said cleansing agent is selected from a group consisting of a liquid, a semi-liquid and a gel; and
i. said cleansing agent is selected from a group consisting of alcohol, an alcohol gel, an antiseptic, hand cleanser and any combination thereof.

65. The dispenser of claim 63, wherein said at least one second product is selected from a group consisting of dry disposable wiping product, wet disposable wiping product, a diaper, a disposable toilet seat cover, a bag, toothpaste, a toothbrush, a razor, a razor blade, a shaving head, a receptacle, a condom, a tampon, ear cleaners, dental floss, a pill, a baby pacifier, talcum powder, cleansing agent, hand lotion, body lotion, makeup, tampons, soap, shampoo, makeup remover, perfume, cologne, toilet water, antiperspirant, deodorant, cash, and tokens; at least one of the following being true:
j. said cleansing agent is selected from a group consisting of a liquid, a semi-liquid and a gel; and
k. said cleansing agent is selected from a group consisting of alcohol, an alcohol gel, an antiseptic, hand cleanser and any combination thereof.

66. The dispenser of claim 63, wherein said at least one adapter and said at least one inner compartment are arranged in a manner selected from a group consisting of concentrically and linearly.

67. The dispenser of claim 63, additionally comprising a hollow lumen configured by means of shape and size to enable said dispenser to be emplaced on either a hanging implement or a support; at least one of the following being true:
l. said hanging implement is selected from a group consisting of be a horizontal rod, a wire, a rope, a string, a hook, a shackle, a suction cup holder and any combination thereof; and
m. said support is selected from a group consisting of a stand, a leg and any combination thereof.

68. The dispenser of claim 63, wherein a material of said dispenser is selected from a group consisting of metal, plastic, silicone, cardboard, glass, paper and any combination thereof; at least one of the following being true:
n. said metal comprises aluminum, iron and any combination thereof;
o. a material of said plastic is selected from a group consisting of rigid, semi-rigid, flexible and any combination thereof; and
p. said plastic is selected from a group consisting of nylon, polyethylene, polyether ether ketone (PEEK), polypropylene, polyvinyl chloride (PVC), polyvinylidene fluoride (PVDF), polycarbonate, polystyrene, nylon, polyethylene terephthalate (PET), polymethylmethacrylate (Acrylic, PMMA), Acetal (Polyoxymethylene, POM), Acrylonitrile Butadiene Styrene (ABS) and any combination thereof.

69. The dispenser of claim 63, wherein at least one of the following is true:
q. a member of a group consisting of said at least one adapter, said compartment, said at least one inner compartment and any combination thereof is refillable; and
r. a member of a group consisting of said at least one adapter, said compartment, said at least one inner compartment and any combination thereof is separable from said dispenser.

70. The dispenser of claim 63, wherein a piped compartment is selected from a group consisting of said external compartment, said at least one inner compartment and any combination thereof; said piped compartment comprising a pipe with a lumen, said pipe providing fluid connection between said piped compartment and an exterior of said device; at least one of the following being true:
s. said pipe is selected from a group consisting of flexible, semi-flexible, rigid and any combination thereof;
t. an external opening of said pipe is selected from a group consisting of flush with the piped compartment, extending beyond a top of the piped compartment, and recessed into the piped compartment;
u. said pipe is either fixed relative to the piped compartment or movable relative to the piped compartment;
v. an exit end of said pipe, when said lid is closed, is flush with the piped compartment and, when said lid is open, said pipe extends beyond the top of the piped compartment;
w. said pipe is extendable beyond the top edge of the piped compartment; and
x. the pipe comprises a member of a group consisting of a lip pullable by a user, an accordion foldable section at its exit end, attachment to the lid such that opening and closing the lid reversibly pulls the exit end of the pipe, a flexible portion such that pulling or pushing on it extrudes the pipe, and any combination thereof.

71. The dispenser of claim 70, wherein the exit end of said pipe is closeable by a conventional sealing closure selected from a group consisting of a cork, a clip-on fitting, a screw-on fitting, a friction-fit fitting, a slide-over fitting, a pinch fitting and any combination thereof at least one of the following being true:
y. said sealing closure is selected from a group consisting of tamper-evident, childproof and any combination thereof;
z. said sealing closure is selected from a member of a group consisting of a pour spout, a trigger, a sprayer cap, a pump, a thumb stop a measuring attachment, a valve, an actuator and any combination thereof;
aa. at least one of said pipe and said sealing closure is configured such that at least one of closure of the exit and pressure on the pipe forces contents of the pipe proximally; and
bb. said sealing closure is selected from a group consisting of tamper-evident, childproof and any combination thereof.

72. The dispenser of claim 63, additionally comprising at least one wing configured by means of size and shape to prevent sliding between said adapter and a member of a group consisting of said roll of dry disposable wiping product, said external compartment, said inner compartment and any combination thereof; said at least one wing having a
configuration selected from a group consisting of longitudinal corrugations, longitudinal ridges, transverse corrugations, transverse ridges, compressible pillows and any combination thereof.
